(19) United States
(12) Patent Application Publication
(10) Pub. No.: US 2025/0006341 A1
(43) Pub. Date: Jan. 2, 2025

(54) HEALTH INFORMATION PLATFORM

(71) Applicant: Pfizer Inc., New York, NY (US)

(72) Inventors: Andreas Panayiotou, Cherry Hills Village, CO (US); David James Ryan, Fort Washington, PA (US); Katherine M. Wietmarschen, Liberty Township, OH (US); Jamie Elizabeth DePeppo, Newksbury Township, NJ (US); John Gabriel Oghia, Hoboken, NJ (US); Galen Chan, Chatham, NJ (US); Dorothy George, Los Angeles, CA (US); Ian Thomas Lyckland, Saunderstown, RI (US); Daniele Codega, Brooklyn, NY (US); Ana Camila Engelbert, Larchmont, NY (US); Rafael Marin Bortolotto, Brooklyn, NY (US); Francesco Bertelli, New York, NY (US); Kathryn Marie Ess, Brooklyn, NY (US); Wynne Kuan Hayes, New York, NY (US); Tim Holt, Nashville, TN (US); Steven Mark Gipstein, San Francisco, CA (US)

(73) Assignee: Pfizer Inc., New York, NY (US)

(21) Appl. No.: 18/754,913

(22) Filed: Jun. 26, 2024

Related U.S. Application Data
(60) Provisional application No. 63/510,546, filed on Jun. 27, 2023.

(51) Int. Cl. G16H 20/60; G06F 3/0488; G16H 70/00

(52) U.S. Cl. CPC G16H 20/60; G06F 3/0488; G16H 70/00

(57) ABSTRACT

A system and methods of providing credibility and trustworthiness to users in a wellness platform. A method for providing search results to a user in an app including searching a query provided in the app, generating a plurality of results of the query, wherein the generating is based on a relevancy of the plurality of results, presenting at least one of the plurality of results in the app to the user, wherein the presenting is based on the relevancy, and displaying, by the at least one processor, the at least one of the plurality of the results in the app as a page, wherein the page has a plurality of sections associated with the at least one of the plurality of results.

### CROSS-REFERENCE TO RELATED APPLICATIONS

The present application claims priority to U.S. Provisional Patent Application No. 63/510,546, titled HEALTH INFORMATION NETWORK WELLNESS PLATFORM, filed Jun. 27, 2023, which is hereby incorporated by reference herein in its entirety.

### TECHNICAL FIELD

Embodiments disclosed herein generally relate to a system and methods of providing credibility and trustworthiness to users in a wellness platform.

### BACKGROUND

Credibility, which refers to the objective and subjective components of the believability of a source or message, are not always easy to come by. Whether the credibility is referring to a business, a news outlet, media, politicians, or science, the credibility is hard to gain back. People are more likely to search and validate the answer on their own than trust those in charge.

Lately, in the world of science, credibility has been a hot topic. Pharmaceutical companies especially are looking for ways to build the trust back or keep the trust they currently have. With the decline in credibility, companies are looking to improve and understand the best way to communicate information to consumers in a reliable and credible way.

### SUMMARY

The present disclosure is directed to a system and method of to a system and methods of providing credibility and trustworthiness to users in a wellness platform.

In one embodiment, the presented disclosure is directed to a computer-implemented method for providing search results to a user in an app, the method comprising: receiving, by at least one processor, a query from a user via the app, wherein the query is associated with a medical disease or condition; in response to the query, generating, by the at least one processor, a result for the query, wherein the result comprises medical information responsive to the query, the medical information comprising a plurality of topics associated with the medical disease or condition; and displaying, by the at least one processor, the result in the app to the user as a plurality of sections as successive screens through which the user can swipe on the app, wherein each of the plurality of sections corresponds to one of the plurality of topics, wherein each of the plurality of sections can be expanded to display additional medical information relevant to the one of the plurality of different topics associated with the corresponding section, wherein each of the plurality of sections comprises a recommendation associated with the corresponding topic.

In one embodiment, the presented disclosure is directed to a computer system for providing search results to a user in an app, the computer system comprising: a processor; and a memory coupled to the processor, the memory storing instructions that, when executed by the processor, cause the computer system to: receive a query from a user via the app, wherein the query is associated with a medical disease or condition; in response to the query, generate a result for the query, wherein the result comprises medical information responsive to the query, the medical information comprising a plurality of topics associated with the medical disease or condition; and display the result in the app to the user as a plurality of sections as successive screens through which the user can swipe on the app, wherein each of the plurality of sections corresponds to one of the plurality of topics, wherein each of the plurality of sections can be expanded to display additional medical information relevant to the one of the plurality of different topics associated with the corresponding section, wherein each of the plurality of sections comprises a recommendation associated with the corresponding topic.

In some embodiments, the plurality of topics comprises nutrition.

In some embodiments, the plurality of sections can be expanded within the app to display additional information regarding the corresponding topic.

In some embodiments, the methods further comprise: displaying, by the at least one processor, a summary of the result for the query.

In some embodiments, the summary comprises at least one of an overview section, a symptoms section, a common causes section, and a recommended next actions section.

In some embodiments, the methods further comprise: providing, by the one or more processor, one or more filters selected by the user; and in response to receiving a selected filter from the one or more filters: generating, by the at least one processor, a result for the query based on the selected filter, and displaying, by the at least one processor, the result in the app.

In some embodiments, he one or more filters comprise at least one of demographics or symptoms.

In some embodiments, the result is generated via a generative machine learning model.

In some embodiments, the generative machine learning model comprises a large language model.

In one embodiment, a method for providing search results to a user in an app includes searching a query provided in the app, generating a plurality of results of the query, wherein the generating is based on a relevancy of the plurality of results, presenting at least one of the plurality of results in the app to the user, wherein the presenting is based on the relevancy, and displaying, the at least one of the plurality of the results in the app as a page, wherein the page has a plurality of sections associated with the at least one of the plurality of results.

In one embodiment, the generating further includes presenting questions to the user based on the query.

In one embodiment, the method further includes expanding the plurality of sections.

In one embodiment, the plurality of sections comprises at least one of paragraphs, lists, nudges, or media.

In one embodiment, the searching is based on the content of the query.

In one embodiment, the query is either a natural language search, a semantic search, or a combination thereof.

### BRIEF DESCRIPTION OF THE FIGURES

The accompanying drawings are incorporated herein and form a part of the specification.

FIGS. 1A-1B depict at least two frameworks used within an app, according to an embodiment of the present disclosure.

FIGS. 2A-2B depict at least two frameworks used within an app, according to an embodiment of the present disclosure.

FIGS. 3A-3C depicts framework A 110 used within an app, according to an embodiment of the present disclosure.

FIGS. 4A-4C depicts framework B 150 used within an app, according to an embodiment of the present disclosure.

FIGS. 5A-5B depicts a query search in the app, according to an embodiment of the present disclosure.

FIGS. 6A-6C depicts a query search in the app where a user is asked additional questions, according to an embodiment of the present disclosure.

FIGS. 7A-7C depicts a query search in the app, according to an embodiment of the present disclosure.

FIG. 8 is a flowchart for a method for implementing a ruleset in a financial institution, according to an embodiment of the present disclosure.

FIG. 9 illustrates an example computer system useful for implementing various embodiments, according to an embodiment of the present disclosure.

FIG. 10 depicts an illustrative search synthesis or summary displayed in response to a search query, according to an embodiment of the present disclosure.

FIG. 11 depicts illustrative filter selections provided to users for tailoring their search query, according to an embodiment of the present disclosure.

### DETAILED DESCRIPTION

The present disclosure is directed to a system and method of providing credibility and trustworthiness to users in a wellness platform.

As previously noted, credibility and trustworthiness are not easy to keep in today's ever changing technological world. Communicating credibility is key to product and business owners to keep the customer or user satisfied. Credibility has certain features that can assist a business in making customers think an item is more credible. These features include, but are not limited to, verification, doctor ratings, the latest findings, transparent discourse, strength of evidence, and user reviews.

In order to increase credibility, a wellness platform, or an application using a

framework can be built. The framework can demonstrate clear and concise content, which allows users to get the information they need easily and quickly, and in a credible way. The framework can be an application, or app, available to the user on their mobile phone or the like. Here, the app can be used in connection with a health information network (HIN) or the like. In the HIN, information regarding disease, diagnosis, or symptoms is available to the user to search and discover.

By using an app, information can be released to the user in a meaningful way in small bits of time, which reduces the overwhelming amount of information available in a given day. The app can also add engagement through a better overall user experience. Additionally, the app can guide users on a path of information that feels logical and relevant to them based on their search. This guidance can provide the credibility on a topic that a user desires.

An app can provide a complimentary content experience that can scale depending on the content topics and production. The app can also provide a transparent discourse, which means all information would not appear immediately to the user, but as they dig deeper into the subject, the information can be provided to the user progressively. The app does allow for short-cuts to specific pages, also referred to as cards, within the app.

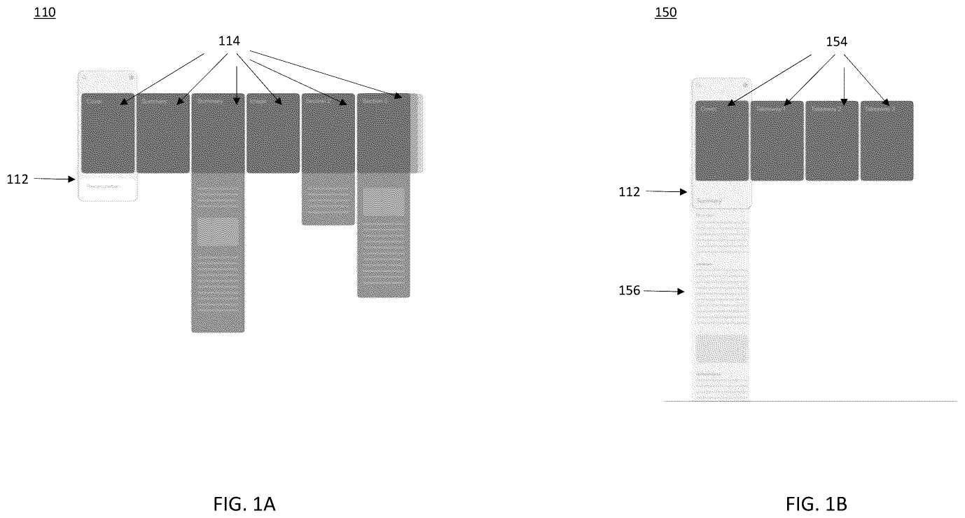

FIGS. 1A and 1B depict at least two frameworks used within an app, according to an embodiment. Within the app, at least two frameworks can exist.

In FIG. 1A, framework A 110 can include at least one card 114 in an app on a mobile phone 112, where each card 114 is a section of the summary of the information and can be expanded. The cards 114 in FIG. 1 are depicted for illustrative purposes and the different sections are visible. In FIG. 1B, Framework B 150 can include key fact cards 154, which can be followed by a long form content page 156.

The cards 114, 154 can include a variety of different information, some including all information possible or summarized content. For example, details on doctors, studies, sources, and alternate viewpoints can be accessible. Doctor verification and user reviews can capture the expertise of a doctor that a user desires to know. The cards 114, 154 can allow more detailed content. Depending on the topic, the user can download all or some of the cards 114, 154. The user can take a journey, or a search, when looking through the cards 114, 154. The cards taken in their totality can serve as building blocks of search across various topics.

Different types of cards 114, 154 can exist such that the user can see the context of the cards 114, 154 and be guided through the summary of the topic. The cards 114, 154 can include paragraphs, media, lists, and nudges. A nudge can refer to a subtle, non-intrusive direction or assistance for the user, which is relevant to the user's context at all times.

The card 114, 154 can also include media as opposed to text. If the card 114, 154 contains media, the media can be video snippets, photos, other visuals, or the like. The card 114, 154 can also suggest local, community, or other businesses that are relevant to the topic. Additionally, the card 114, 154 can provide verification, such as by doctors or that the topic has been medically reviewed. This verification can add to the credibility of the topic. The card 114, 154 can include the source of the information on the topic as well, which can provide further credibility to the topic on the card 114, 154.

When searching and building cards 114, 154, three key components can be considered: content, query, and ingredients. Content can refer to what content exists and how to surface existing content. Query can refer to any of the following: how queries are matched to the available content, the combination of either a natural language search or a semantic search, or how the users can be guided to better search queries. Ingredients can refer to how the different pieces of the experience fit together, how to showcase the different content types, and how elements, such as personalization, affect the search. The cards 114, 154 can focus on how the search experience will feel to the user, not necessarily on the inputs of the search by the user.

Framework A 110 and framework B 150 can guide a user intuitively through the cards 114, 154, respectively. By guiding the user, the cards 114, 154 give the user the right level of information and at the right moment in their search of information. Both frameworks 110, 150 also allow the user to go through more concise information if necessary. However, the difference in the frameworks 110, 150 is how the extended subjects are revealed. The difference in the way the cards 114, 154 expand in one framework versus the other may resonate better with a user.

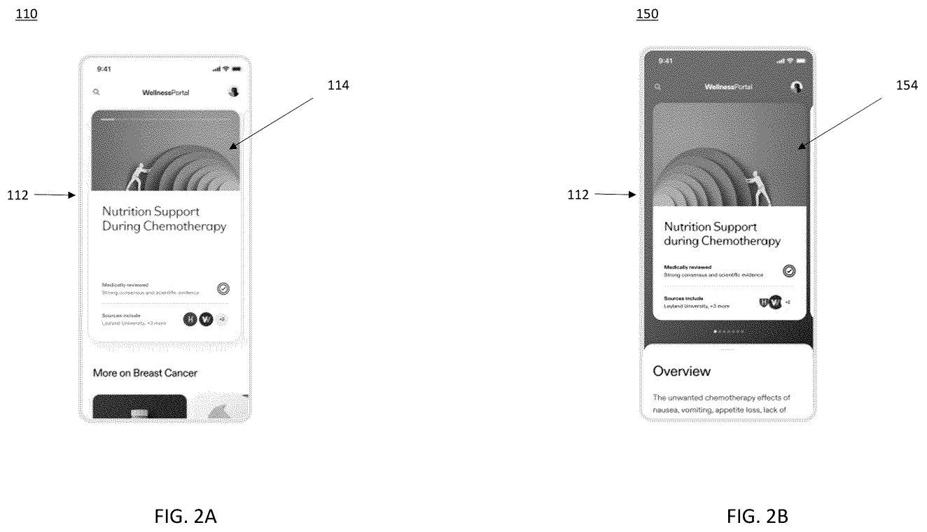

FIGS. 2A and 2B depict at least two frameworks used within an app, according to an embodiment.

FIG. 2A depicts framework A 110 in an app on a mobile phone 112. Within the framework A 110, the card 114 is a section of the summary and can be expanded. FIG. 2B depicts framework B 150 in an app on a mobile phone 112. Within the framework B 150, the card 154 is a key fact card that has a long form content page.

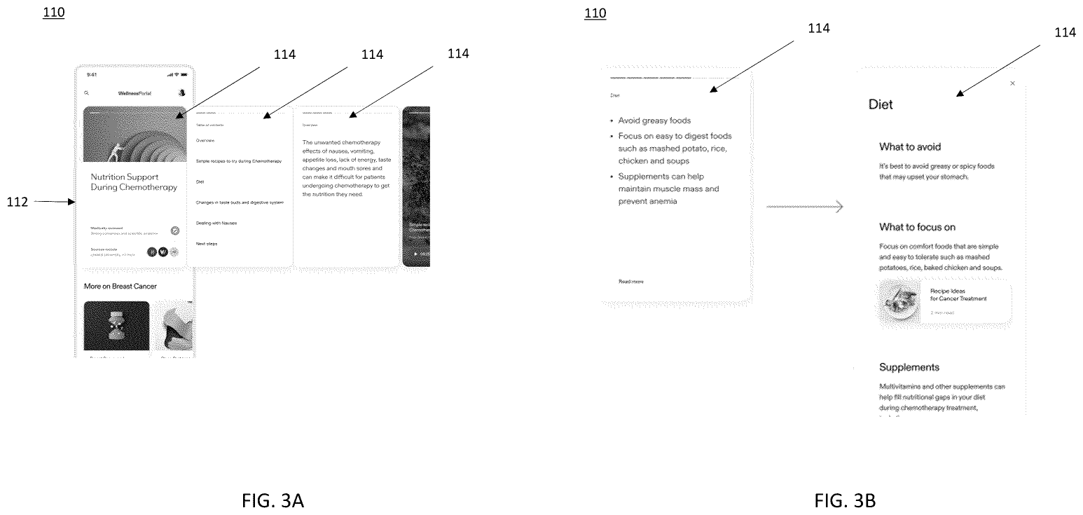

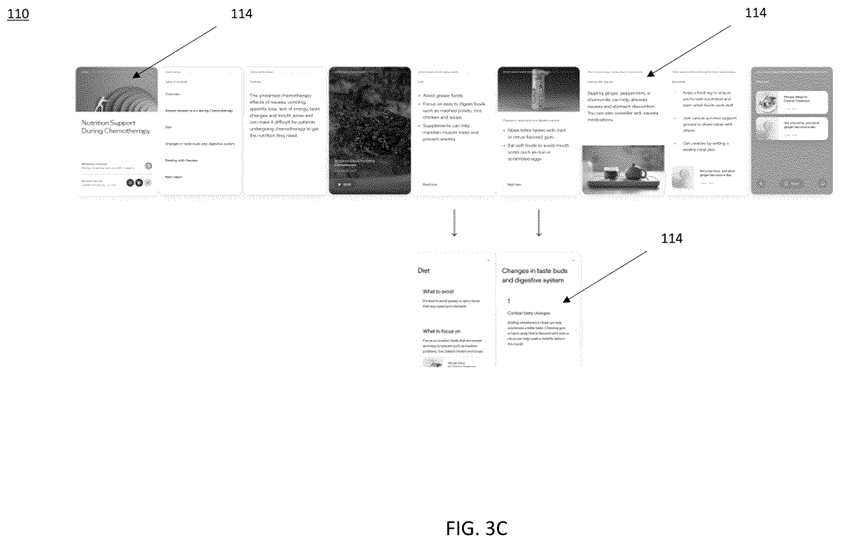

FIGS. 3A-3C depicts framework A 110 used within an app, according to an embodiment.

FIG. 3A depicts framework A 110 in an app on a mobile phone 112. Within the framework A 110, the card 114 can organize summary content by section. For example, if the topic of the card 114 is nutrition during chemotherapy, the cards 114 can expand based on the sections.

FIG. 3B depicts framework A 110 in an app. Within the framework A 110, the card 114 can be expanded for further reading, based on whether the user thinks further reading is necessary. As described, the topic of the card 114 is nutrition during chemotherapy. One of the sections of the card 114 that can be expanded is regarding diet.

FIG. 3C depicts framework A 110 in an app. Within the framework A 110, each section of the topic can get one card 114. As described, the topic of the card 114 is nutrition during chemotherapy. The information on the card 114 can be provided progressively or jumped to, based on the desire of the user.

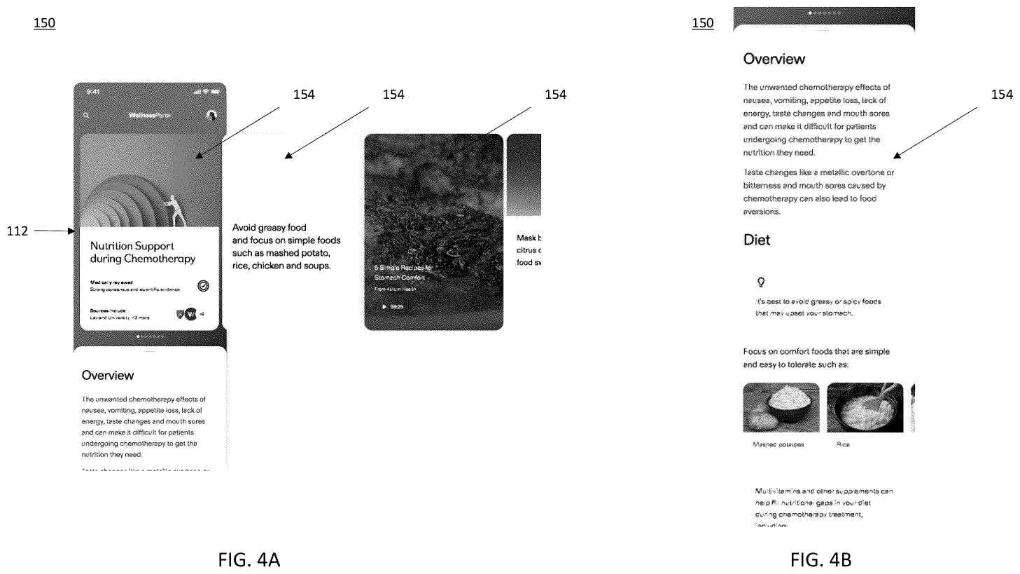

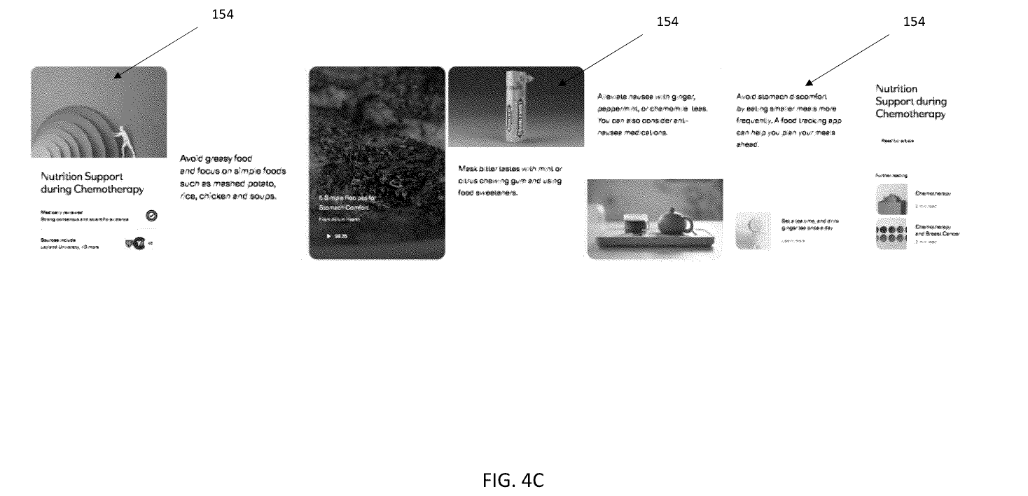

FIGS. 4A-4C depicts framework B 150 used within an app, according to an embodiment.

FIG. 4A depicts framework B 150 in an app on a mobile phone 112. Within the framework B 150, the card 154 can highlight key facts, which can then by followed by all the information. For example, if the topic of the card 154 is nutrition during chemotherapy, the cards 154 have highlights of nutrition.

FIG. 4B depicts framework B 150 in an app, Within the framework B 150, the card 154 allows the user to scroll down and the user can choose to read or see all the information about a specific section. For example, if the topic of the card 154 is nutrition during chemotherapy, the user can click on the card 154 and choose to read material on this topic.

FIG. 4C depicts framework B 150 in an app. Within the framework B 150, the cards 154 form their own standalone story of the topic. For example, if the topic of the card 154 is nutrition during chemotherapy, the card 154 forms a story about food and diet and how to get nutrition support during chemotherapy. The information on the card 154 can be provided progressively or jumped to, based on the desire of the user.

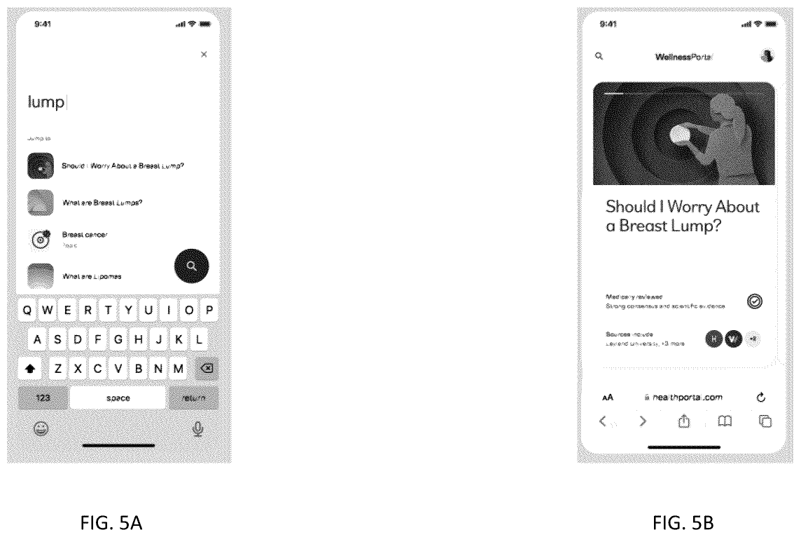

FIGS. 5A-5B depicts a query search in the app, according to an embodiment.

In FIG. 5A, the user may search in the search bar on a search form. The user can find content or topics that matches their intent within the top suggestions as they type and content that matches exactly what they are searching for. For example, a user may wonder about “User is concerned about a newly found lump in their breast,” and want to search. In the search bar of the app, the user may enter only the word “lump” based on their desire. After searching, the app can return different sections and topics, such as “should I worry about a breast lump,” “what are breast lumps,” “breast cancer,” or “what are lipomas.”

In FIG. 5B, the user chooses the content that they are searching for to see a summary page. The user can choose from the search form in FIG. 5A and then jump into a summary page of the given topic or section. Specifically, the user can choose the first topic “should I worry about a breast lump.” Once choosing, the app jumps to the summary page of the topic. The user can then read at their leisure about the topic.

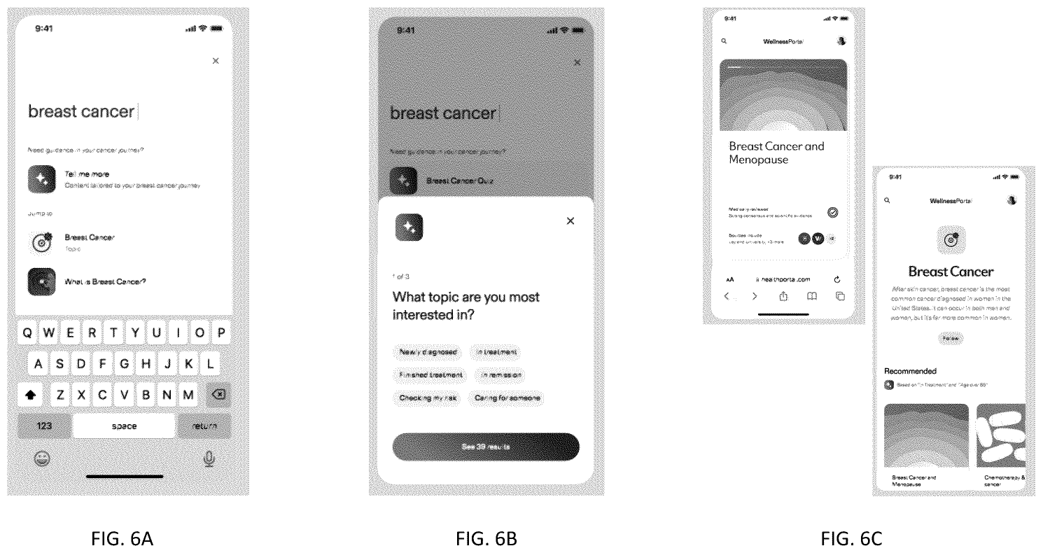

FIGS. 6A-6C depict a query search in the app where a user is asked additional questions, according to an embodiment.

In FIG. 6A, the user can search in a search bar on a search form. Occasionally, depending on the topic of the search, the app can ask the user additional questions. The user can search a specific topic based on a topic they are looking for more information on. For example, a user wants to query something after they have “just been diagnosed with breast cancer and is looking for more information.” They can type “breast cancer” into the search bar, as depicted in FIG. 6A.

In FIG. 6B, the user can be provided with additional questions based on their search. The user can be asked a few questions to personalize the content they will see next. For example, the user has searched for “breast cancer.” Since that is a broad topic, the app can ask a few questions, such as a more specific topic related to breast cancer. Questions can include whether the user is “newly diagnosed,” “in treatment,” “finished treatment,” “in remission,’ “checking my risk,” or “caring for someone.”

In FIG. 6C, the user can be taken to a personalized topic based on their answer to the questions. The user can see topics that prioritize content based on the matches to the questions. For example, the user has answered questions regarding their search of breast cancer. The user is then given a personalized topic of “breast cancer and menopause” or the topic page of “breast cancer.”

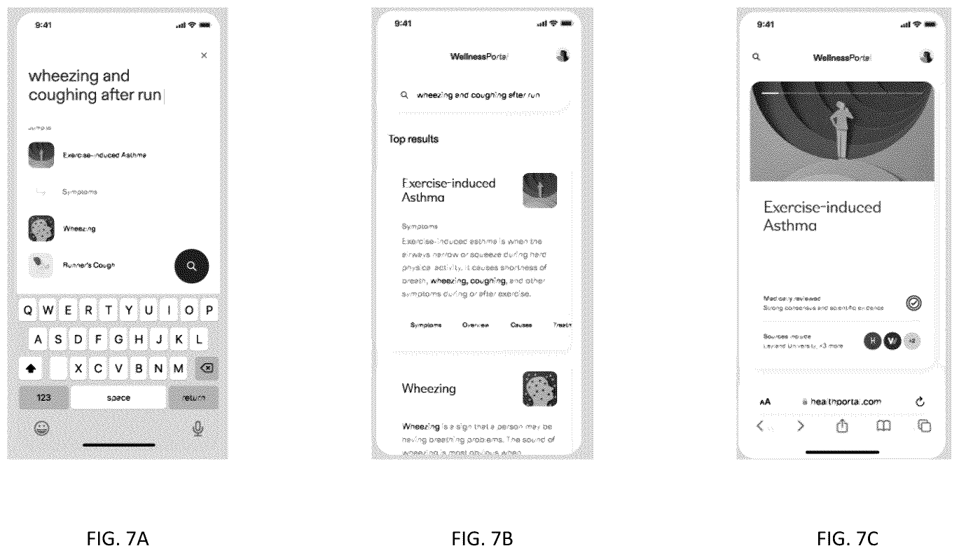

Occasionally, the app will provide topics to a user's query that the user isn't sure if the suggested topics are what they are searching for. The user can then decide to explore more results, as depicted in FIG. 7.

FIGS. 7A-7C depict a query search in the app, according to an embodiment.

In FIG. 7A, the user can search in a search bar on a search form. If the user isn't confident in the suggestions of the search, the user can choose to go to a full search results page. For example, a user can search for “wheezing and coughing after a run.” The user can choose one of the content pages of the topics to jump to.

In FIG. 7B, the user can see additional snippets of the content for each result. Each result of the topic can help the user differentiate between the different pieces of content and decide what to read next. For example, the user can see a snippet of the topics in the search for “wheezing and coughing after a run.” The topics, such as “exercise-induced asthma” or “wheezing”, provide a snippet for the user to understand the topic more.

In FIG. 7C, the user can see the summary page of the chosen topic. Based on the snippet, the user has chosen the topic they are interested in. The user can then jump to the summary page of that topic. This summary page is a part of the card. For example, the user has chosen “exercise-induced asthma” and is taken to the summary page of the topic. The user can stay on the summary page of the card or begin a deeper search within that topic and search further cards.

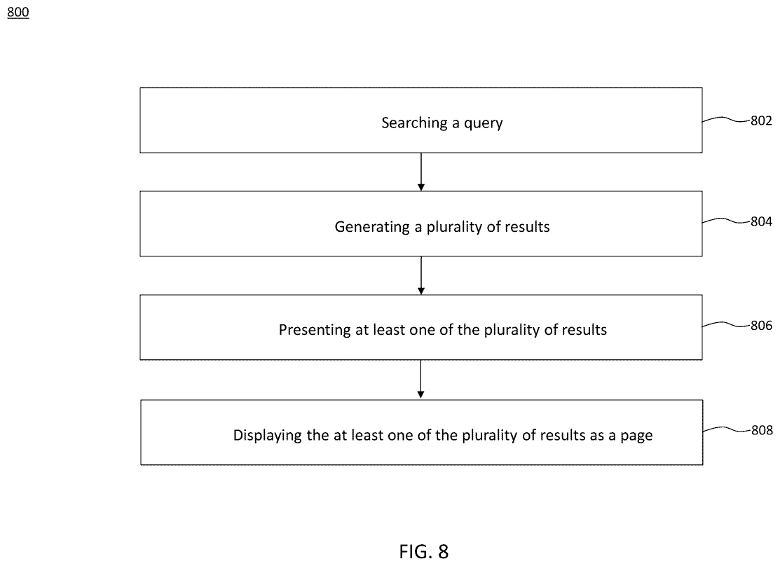

FIG. 8 is a flowchart for a method 800 for providing credibility and trustworthiness to users in a wellness platform, according to an embodiment. Method 800 can be performed by processing logic that can comprise hardware (e.g., circuitry, dedicated logic, programmable logic, microcode, etc.), software (e.g., instructions executing on a processing device), or a combination thereof. It is to be appreciated that not all steps can be needed to perform the disclosure provided herein. Further, some of the steps can be performed simultaneously, or in a different order than shown in FIG. 8, as will be understood by a person of ordinary skill in the art.

Method 800 shall be described with reference to FIGS. 1-7. However, method 800 is not limited to that example embodiment.

In 802, a query provided in an app can be searched. The searching can be based on the content of the query. The query can be either a natural language search, a semantic search, or a combination thereof. For example, in an app on a mobile phone 112, a query can be provided in a search form. As depicted in FIGS. 5-7, a query can be provided such as “lump”, “breast cancer,” or “wheezing and coughing after run.”

In 804, a plurality of results of the query can be generated. The generated results can be based on a relevancy of the plurality of results. The generating can further include presenting questions to the user based on the query. For example, in the app on the mobile phone 112, the results of the query can be generated. As depicted in FIGS. 5-7, results of the query are generated. In response to “wheezing and coughing after run,” the result of “exercise-induced asthma” or “wheezing” can be generated.

In 806, at least one of the plurality of results in the app can be presented. The at least one presented result can be based on the relevancy. For example, in the app on the mobile phone 112, the results are presented. As depicted in FIGS. 5-7, “exercise-induced asthma” is presented to the user.

In 808, the at least one of the plurality of results as a page can be displayed. The page can have a plurality of sections associated with the at least one of the plurality of results. The method can include expanding the plurality of sections. The plurality of sections can include at least one of paragraphs, lists, nudges, or media. For example, in the app on the mobile phone 112, the card 114, 154 is displayed. The cards 114, 154 can be expanded based on the user's desires.

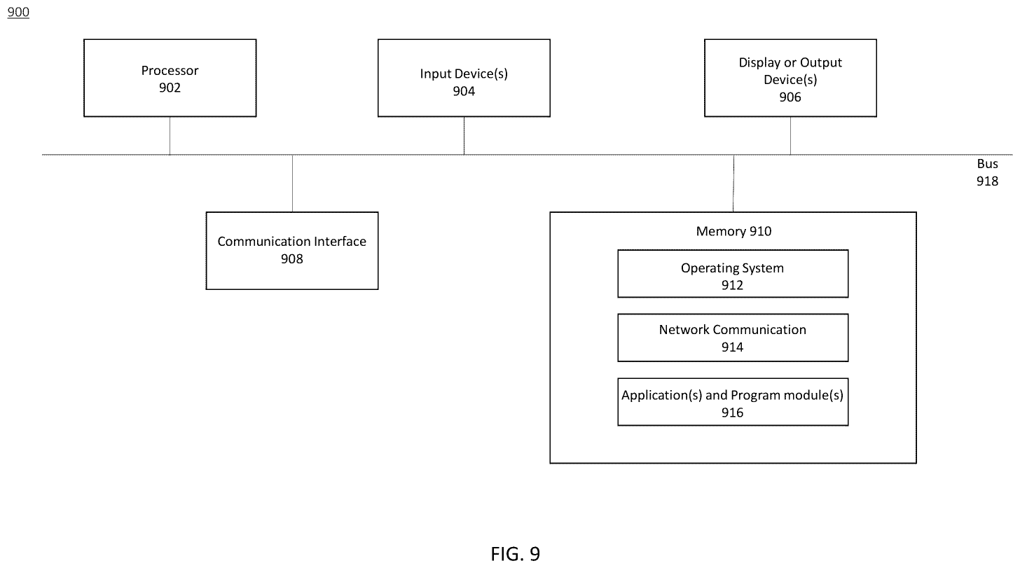

FIG. 9 is a block diagram of an example computing device 900 that may be utilized to execute embodiments to implement processes including various features and functional operations as described herein. For example, computing device 900 may function as framework A 110 or framework B 150 or a portion or combination thereof. The computing device 900 may be implemented on any electronic device to execute software applications derived from program instructions for and as illustrated in FIGS. 1-8, and includes but not limited to personal computers, servers, smartphones, media players, electronic tablets, game consoles, email devices, etc. In some implementations, the computing device 900 may include one or more processors 902, one or more input devices 904, one or more display devices or output devices 906, one or more communication interfaces 908, and memory 910. Each of these components may be coupled by bus 918, or in the case of distributed computer systems, one or more of these components may be located remotely and accessed via a network.

Processor(s) 902 may use any known processor technology, including but not limited to graphics processors and multi-core processors. Suitable processors for the execution of a program of instructions may include, by way of example, both general and special purpose microprocessors, and the sole processor or one of multiple processors or cores, of any kind of computer. Generally, a processor may receive instructions and data from a read-only memory or a random-access memory or both. The essential elements of a computer may include a processor for executing instructions and one or more memories for storing instructions and data. Generally, a computer may also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data may include all forms of non-transitory memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory may be supplemented by, or incorporated in, ASICs (application-specific integrated circuits).

Input devices 904 may be any known input devices technology, including but not limited to a keyboard (including a virtual keyboard), mouse, track ball, and touch-sensitive pad or display. To provide for interaction with a user, the features and functional operations described in the disclosed embodiments may be implemented on a computer having a display device 906 such as a CRT (cathode ray tube) or LCD (liquid crystal display) monitor for displaying information to the user and a keyboard and a pointing device such as a mouse or a trackball by which the user can provide input to the computer. Display device 906 may be any known display technology, including but not limited to display devices using Liquid Crystal Display (LCD) or Light Emitting Diode (LED) technology.

Communication interfaces 908 may be configured to enable computing device 900 to communicate with other another computing or network device across a network, such as via a wired connection, a wireless connection, or a combination of wired and wireless connections. For example, communication interfaces 908 may include an Ethernet interface, an optical interface, a coaxial interface, an infrared interface, a radio frequency (RF) interface, a universal serial bus (USB) interface, a Wi-Fi interface, a cellular network interface, or the like.

Memory 910 may be any computer-readable medium that participates in providing computer program instructions and data to processor(s) 902 for execution, including without limitation, non-transitory computer-readable storage media (e.g., optical disks, magnetic disks, flash drives, etc.), or volatile media (e.g., SDRAM, ROM, etc.). Memory 910 may include various instructions for implementing an operating system 912 (e.g., Mac OS®, Windows®, Linux). The operating system may be multi-user, multiprocessing, multitasking, multithreading, real-time, and the like. The operating system may perform basic tasks, including but not limited to: recognizing inputs from input devices 904; sending output to display device 906; keeping track of files and directories on memory 910; controlling peripheral devices (e.g., disk drives, printers, etc.) which can be controlled directly or through an I/O controller; and managing traffic on bus 918. Bus 918 may be any known internal or external bus technology, including but not limited to ISA, EISA, PCI, PCI Express, USB, Serial ATA or Fire Wire.

Network communications instructions 914 may establish and maintain network connections (e.g., software applications for implementing communication protocols, such as TCP/IP, HTTP, Ethernet, telephony, etc.). Application(s) and program modules 916 may include software application(s) and different functional program modules which are executed by processor(s) 902 to implement the processes described herein and/or other processes. For example, the program modules 916 may include a service management module for retrieving features associated with user transactions described herein for accessing program components and application processes. The program modules 916 may include but not limited to software programs, machine learning models, objects, components, data structures that are configured to perform tasks or implement the processes described herein. The processes described herein may also be implemented in operating system 912.

In practice, a health information or wellness platform in accordance with the subject matter described herein may include summary or synthesis generated by artificial intelligence (AI) tools or sources, including machine learning models and/or algorithms. The synthesis generated by the AI tools or sources may include a summary of key findings across credible sources based on a user's search. In some embodiments, the system could implement large language models (LMMs) for generating the summaries or syntheses. In various embodiments, the machine learning models and/or algorithms may include neural networks, decisions trees (e.g., random forests), support vector machines, regressions, hidden Markov models, and other types of machine learning techniques known in the field. Further, the neural networks may include any general category of neural network, including deep neural networks, convolutional neural networks, autoencoders, recurrent neural networks, and so on. Further, the machine learning models described herein may be trained using supervised or unsupervised learning techniques.

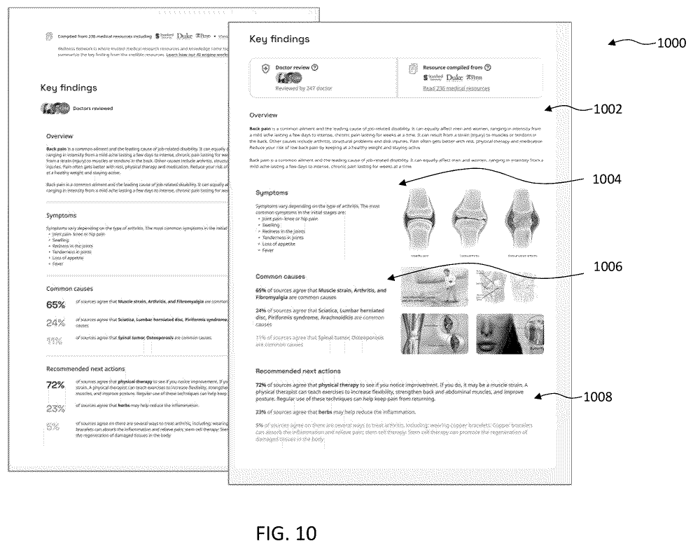

Referring now to FIG. 10 where an illustrative search synthesis or summary 1000 is presented herein for illustrative purposes. In some embodiments, a summary of key findings across sources pertaining to a user's search based on keywords and filters may be presented to the user with a showing of a list of source articles. As shown, the summary or synthesis may have an overview section 1002 outlining information such as what a particular illness or condition is about; a symptoms section 1004 illustrating a list of accompanying symptoms, sometimes with the corresponding drawings; a common causes section 1006 explaining some of the possible causes for the symptoms; and a recommended next actions section 1008 to provide the user with some follow up actions. In some embodiments, all these sections may be generated using an AI tool or algorithm, such as a generative AI model or algorithm. In various embodiments, the generative AI models or algorithms implemented by the system could include Bayesian networks, diffusion models, generative adversarial networks, variation autoencoders, Markov chains, and any other models and/or algorithms utilized in the generative AI field. In some embodiments, predicted searches pointing to a directory of topics with pre-generated AI summaries, stored in a database may be provided, where summaries may be pre-generated by AI based on highest priority and most popular search terms and phrases. And filtering may bring up pre-generated summaries with those terms and links to source articles.

In some embodiments, the recommended next actions 1008 may be generated by AI. In one embodiment, the recommended actions 1008 could be generated using LLMs. In other embodiments, the recommend actions 1008 could be generated using the Bayesian networks, diffusion models, generative adversarial networks, variation autoencoders, Markov chains, and any other models and/or algorithms utilized in the generative AI field. Such AI-based recommendation can assist users on what to do for self-care or seeking care. The user may sec potential next actions one can take in the summary page 1000 which includes popular next steps based on the source articles. Furthermore, in some other embodiments, the health information or wellness platform may provide users access to external telehealth and Health Care Professional (HCP) booking services, as well as links to content for recommendations (e.g., “click here to learn more”).

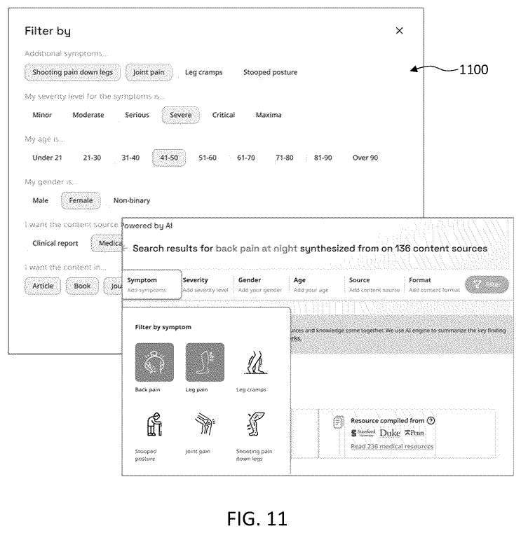

In some embodiments, guided search filters may be utilized to improve the searching performance. Referring now to FIG. 11, a user can add filters 1100 to their search to clarify symptoms or narrow in on demographics at the search bar or once on their summary page, and subsequently receive a summary page that is tailored based on their filters. In some embodiments, quantity of filters may depend on tagging of source articles (i.e., content source, people to tag, AI to scrape and tag). In some other embodiments, user may select context-specific filters (e.g., symptoms for one issue), and summaries with relevant tags from the filter may be shown. In yet another embodiment, a list of filters may be made available based on user experiences or feedback.

Furthermore, credible institutions or experts may be consulted to provide credibility of the cited articles or information, and visual markers may be provide to illustrate such credibility. For example, credible institution tag may be presented visually on each summary, based on the meta data of source articles.

The features and functional operations described in the disclosed embodiments may be implemented in one or more computer programs that may be executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program may be written in any form of programming language (e.g., Objective-C, Java), including compiled or interpreted languages, and it may be deployed in any form, including as a stand-alone program or as a module, component, subroutine, or other unit suitable for use in a computing environment.

The described features and functional operations described in the disclosed embodiments may be implemented in a computer system that includes a back-end component, such as a data server, or that includes a middleware component, such as an application server or an Internet server, or that includes a front-end component, such as a user device having a graphical user interface or an Internet browser, or any combination thereof. The components of the system may be connected by any form or medium of digital data communication such as a communication network. Examples of communication networks include, e.g., a telephone network, a LAN, a WAN, and the computers and networks forming the Internet.

The computer system may include user computing devices and application servers. A user computing device and server may generally be remote from each other and may typically interact through a network. The relationship of user computing devices and application server may arise by virtue of computer programs running on the respective computers and having a client-server relationship to each other.

Communication between various network and computing devices 900 of a computing system may be facilitated by one or more application programming interfaces (APIs). APIs of system may be proprietary and/or may be examples available to those of ordinary skill in the art such as Amazon® Web Services (AWS) APIs or the like. The API may be implemented as one or more calls in program code that send or receive one or more parameters through a parameter list or other structure based on a call convention defined in an API specification document. One or more features and functional operations described in the disclosed embodiments may be implemented using an API. An API may define one or more parameters that are passed between an application and other software instructions/code (e.g., an operating system, library routine, function) that provides a service, that provides data, or that performs an operation or a computation. A parameter may be a constant, a key, a data structure, an object, an object class, a variable, a data type, a pointer, an array, a list, or another call.

While various embodiments have been described above, it should be understood that they have been presented by way of example and not limitation. It will be apparent to persons skilled in the relevant

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments can be used, and other changes can be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Instead, this application is intended to cover any variations, uses, or adaptations of the present teachings and use its general principles. Further, this application is intended to cover such departures from the present disclosure as come within known or customary practice in the art to which these teachings pertain. Many modifications and variations can be made to the particular embodiments described without departing from the spirit and scope of the present disclosure as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

Various of the above-disclosed and other features and functions, or alternatives thereof, can be combined into many other different systems or applications. Various presently unforeseen or unanticipated alternatives, modifications, variations or improvements therein can be subsequently made by those skilled in the art, each of which is also intended to be encompassed by the disclosed embodiments.

As used in this document, the singular forms “a,” “an,” and “the” include plural references unless the context clearly dictates otherwise. With respect to the use of substantially any plural and/or singular terms herein, those having skill in the art can translate from the plural to the singular and/or from the singular to the plural as is appropriate to the context and/or application. The various singular/plural permutations can be expressly set forth herein for sake of clarity.

As will be understood by one skilled in the art, for any and all purposes, such as in terms of providing a written description, all ranges disclosed herein are intended as encompassing each intervening value between the upper and lower limit of that range and any other stated or intervening value in that stated range. All ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells as well as the range of values greater than or equal to 1 cell and less than or equal to 3 cells. Similarly, a group having 1-5 cells refers to groups having 1, 2, 3, 4, or 5 cells, as well as the range of values greater than or equal to 1 cell and less than or equal to 5 cells, and so forth.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, structures, or groups thereof, or any members of a claimed group, less than the full measure of this disclosure can be claimed for any reason.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art. Where the context of the disclosure indicates otherwise, or is inconsistent with such an interpretation, the above-stated interpretation can be modified as would be readily apparent to a person skilled in the art. For example, in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). Further, the transitional term “comprising,” which is synonymous with “including,” “containing,” or “characterized by,” is inclusive or open-ended and does not exclude additional, unrecited elements or method steps. While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

Throughout this disclosure, various patents, patent applications and publications can be referenced. The disclosures of these patents, patent applications and publications are incorporated into this disclosure by reference in their entireties in order to more fully describe the state of the art as known to those skilled therein as of the date of this disclosure. This disclosure will govern in the instance that there is any inconsistency between the patents, patent applications and publications cited and this disclosure.

Additional details, description, drawings and embodiments for the herein described health information network platform can be found in the attached appendix.

1. A computer-implemented method for providing search results to a user in an app, the method comprising:
receiving, by at least one processor, a query from a user via the app, wherein the query is associated with a medical disease or condition;
in response to the query, generating, by the at least one processor, a result for the query, wherein the result comprises medical information responsive to the query, the medical information comprising a plurality of topics associated with the medical disease or condition; and
displaying, by the at least one processor, the result in the app to the user as a plurality of sections as successive screens through which the user can swipe on the app, wherein each of the plurality of sections corresponds to one of the plurality of topics, wherein each of the plurality of sections can be expanded to display additional medical information relevant to the one of the plurality of different topics associated with the corresponding section, wherein each of the plurality of sections comprises a recommendation associated with the corresponding topic.

2. The computer-implemented method of claim 1, wherein the plurality of topics comprises nutrition.

3. The computer-implemented method of claim 1, wherein the plurality of sections can be expanded within the app to display additional information regarding the corresponding topic.

4. The computer-implemented method of claim 1, further comprising:
displaying, by the at least one processor, a summary of the result for the query.

5. The computer-implemented method of claim 4, wherein the summary comprises at least one of an overview section, a symptoms section, a common causes section, and a recommended next actions section.

6. The computer-implemented method of claim 1, further comprising:
providing, by the one or more processor, one or more filters selected by the user; and
in response to receiving a selected filter from the one or more filters:
generating, by the at least one processor, a result for the query based on the selected filter, and
displaying, by the at least one processor, the result in the app.

7. The computer-implemented method of claim 6, wherein the one or more filters comprise at least one of demographics or symptoms.

8. The computer-implemented method of claim 1, wherein the result is generated via a generative machine learning model.

9. The computer-implemented method of claim 8, wherein the generative machine learning model comprises a large language model.

10. A computer system for providing search results to a user in an app, the computer system comprising:
a processor; and
a memory coupled to the processor, the memory storing instructions that, when executed by the processor, cause the computer system to:
receive a query from a user via the app, wherein the query is associated with a medical disease or condition;
in response to the query, generate a result for the query, wherein the result comprises medical information responsive to the query, the medical information comprising a plurality of topics associated with the medical disease or condition; and
display the result in the app to the user as a plurality of sections as successive screens through which the user can swipe on the app, wherein each of the plurality of sections corresponds to one of the plurality of topics, wherein each of the plurality of sections can be expanded to display additional medical information relevant to the one of the plurality of different topics associated with the corresponding section, wherein each of the plurality of sections comprises a recommendation associated with the corresponding topic.

11. The computer system of claim 10, wherein the plurality of topics comprises nutrition.

12. The computer system of claim 10, wherein the plurality of sections can be expanded within the app to display additional information regarding the corresponding topic.

13. The computer system of claim 10, wherein the memory stores further instructions that, when executed by the processor, cause the computer system to:
display a summary of the result for the query.

14. The computer system of claim 13, wherein the summary comprises at least one of an overview section, a symptoms section, a common causes section, and a recommended next actions section.

15. The computer system of claim 10, wherein the memory stores further instructions that, when executed by the processor, cause the computer system to:
provide one or more filters selected by the user; and
in response to receiving a selected filter from the one or more filters:
generate a result for the query based on the selected filter, and
display the result in the app.

16. The computer system of claim 15, wherein the one or more filters comprise at least one of demographics or symptoms.

17. The computer system of claim 10, wherein the result is generated via a generative machine learning model.

18. The computer system of claim 17, wherein the generative machine learning model comprises a large language model.
